(19) United States
(12) Patent Application Publication
(10) Pub. No.: US 2017/0258630 A1
(43) Pub. Date: Sep. 14, 2017

(54) OPERATING ROOM (OR) TABLE COVER

(71) Applicant: Revolutionary Medical Devices, Inc., Tucson, AZ (US)

(72) Inventors: Michael J. Pedro, Windham, NH (US); Steven H. Cataldo, New York, NY (US); David M. Kane, Tucson, AZ (US); Thomas M. Reilly, Tucson, AZ (US); James Ellis, Tempe, AZ (US); Peter Krones, Katonah, NY (US)

(21) Appl. No.: 15/532,021

(22) PCT Filed: Dec. 10, 2015
(86) PCT No.: PCT/US15/65105

§ 371 Date: May 31, 2017

Related U.S. Application Data
(60) Provisional application No. 62/090,173, filed on Dec. 10, 2014.
(60) Provisional application No. 62/135,045, filed on Mar. 18, 2015.
(60) Provisional application No. 62/141,640, filed on Apr. 1, 2015.

(51) Int. Cl. A61F 7/00; A61G 13/10; A61G 13/12

(52) U.S. Cl. CPC A61F 7/0053; A61G 13/121; A61G 13/101; A61F 7/007; A61F 2007/006; A61F 2007/0095; A61F 2007/0071

(57) ABSTRACT

An OR table disposable cover includes a sheet, fluid absorbent on a top side, and fluid impermeable on an opposite, bottom side; a angled support wedge positioned in a head support region on the top side of the sheet; and a soft foam ring member on a top of the angled support which in concert with its underlying support wedge maintains a patient's head in a sniff position. Also provided is a barrier cover having a patient head support member adjacent one end, said head support member being canted at an about 15° angle to the cover, and an operating table carriage that interfaces with the patient either directly, or through a cushion that allows the patient's head to translate along an Y axis of the rail.

The present invention in one aspect relates to improvements in operating room (OR) table covers, and more particularly to an OR table cover for an OR table designed to facilitate the maintenance of a patent airway while the patient is either unconscious, unable to maintain a patent airway, or under sedation and/or anesthesia. In another aspect, the present invention relates to an OR table cover which incorporates a head positioning support to facilitate the maintenance of a patent airway, and also incorporating means for patient warming. The present invention also relates to improvements in head supports for operating tables and an operating table carriage that interfaces with the patient either directly or through a cushion that allows the patient's head to translate along the Y axis of the rail, while facilitating the maintenance of a patent airway.

In our PCT Application Serial No. PCT/US14/44934, we describe positioning devices for positioning and supporting a patient while the patient is either unconscious, unable to maintain a patent airway or under sedation and/or anesthesia to facilitate the maintenance of a patent airway by correctly positioning a patient's back, shoulders, neck and head using an adjustable ramp with placement of the patient's back and shoulders to achieve a desired angle, and a head support for optimizing the sniffing position. Also, in our PCT Application Serial No. PCT/US15/45357, we describe a patient head support system for positioning a patient to facilitate maintenance of a patent airway comprising a head support pillow and a head support where the head support pillow comes into contact with the patient's head and/or neck on one side and the other side is in contact and can either be attached or detached from a pillow support on the OR table, wherein the pillow and pillow support allow for approximately 15° of head extension to place the patient in the desired “sniffing position”.

The present invention provides improvements in OR table covers, particularly but not limited for use with OR tables incorporating patient positioning devices such as described in our aforesaid PCT/US14/44934 application, and in our aforesaid PCT/US15/45357.

In one aspect of the invention there is provided an OR table disposable cover, comprising: a sheet, fluid absorbent on a top side, and fluid impermeable on an opposite, bottom side; a angled support wedge positioned in a head support region on the top side of the sheet; and a soft foam ring member on a top of the angled support which in concert with its underlying support wedge maintains a patient's head in a sniff position.

In one preferred aspect of the invention the angle support wedge and foam ring maintains a patient's head at approximately 15° angle relative to the OR table.

In another preferred aspect of the invention the angled support wedge is formed of a rigid foam.

In yet another aspect of the invention, the cover includes a patient warming device integrated with the OR table cover, said table cover including a plenum supplied with heated air from a thermally controlled air source and exiting a surface between the plenum and the patient. Preferably the thermally-controlled heat source operates in open loop, or a closed loop with temperature feedback provided by temperature sensors located between the patient and plenum or at the thermally-controlled heat source.

In still yet another aspect of the invention, the cover includes a patient warming device integrated with the OR table disposable cover, said warming device including a resistive grid. Preferably the resistive grid operates in open loop, or a closed loop with temperature feedback provided by temperature sensors located between the patient and electrical an current or voltage source.

The present invention also provides a protective barrier cover for placement between the OR and the patient, said banner cover having a removable head cover section adjacent one end, and indicia for determining approximate patient height and for guiding location of a patient on a support surface.

In one embodiment the removable head cover section has one or more pre-cut or perforated apertures for permitting access to a head support system for permitting attachment of mask anchor and/or head anchor straps such as described in our aforesaid PCT/US14/44934 application or our PCT/US15/34277 application.

In another embodiment, the removable head cover section is perforated so that it may be punched out and moved elsewhere on the barrier cover.

In one embodiment the barrier cover includes indicia with unit of length markings, e.g., Imperial, U.S. and/or metric markings and/or cautionary markings to alert the staff to a possible pinching hazard.

In another embodiment the removable head cover has one or more pre-cut anesthesia head strap attachment mechanism sections, which allow the head cover to surround an anesthesia head strap attachment mechanism.

In yet another embodiment, the pre-cut sections allow anesthesia head strap attachment mechanisms to be visible and used to attach to head straps.

In yet another embodiment there is provided a protective barrier for placement between the OR and the patient, which includes a support for placing the patient's head in the sniff position.

In one embodiment, the support for placing the patient's head in the sniff position comprises a head support ring formed of a resiliently deformable material such as foam. The support ring may be formed with an approximately 15° wedge angle, or a separate approximately 15° wedge may be placed under the support ring for placing the patient's head in a sniff position.

In still yet another embodiment, the bottom of the covering has openings to allow for slide pin mating holes for adjustment of the support ring.

In still yet another embodiment, the head support ring may be positioned and held in place by hook and loop fasteners.

The present invention also provides a system for controlling the translation of a patient's head along a head support plane with a rail attached to the head support plane and a carriage that slides along the rail as will be described below.

In one aspect, the present invention provides a rail or carriage that is attached to an operating table head support, constraining a carriage in the X and Z axes, allowing a carriage to travel along the rail in a Y axis.

In another aspect the present invention provides a rail or carriage as above described, further comprising a brake that when engaged, holds the carriage in position on the rail.

Features and advantages of the present invention will be seen from the following detailed description, taken in conjunction with the accompanying drawings, wherein:

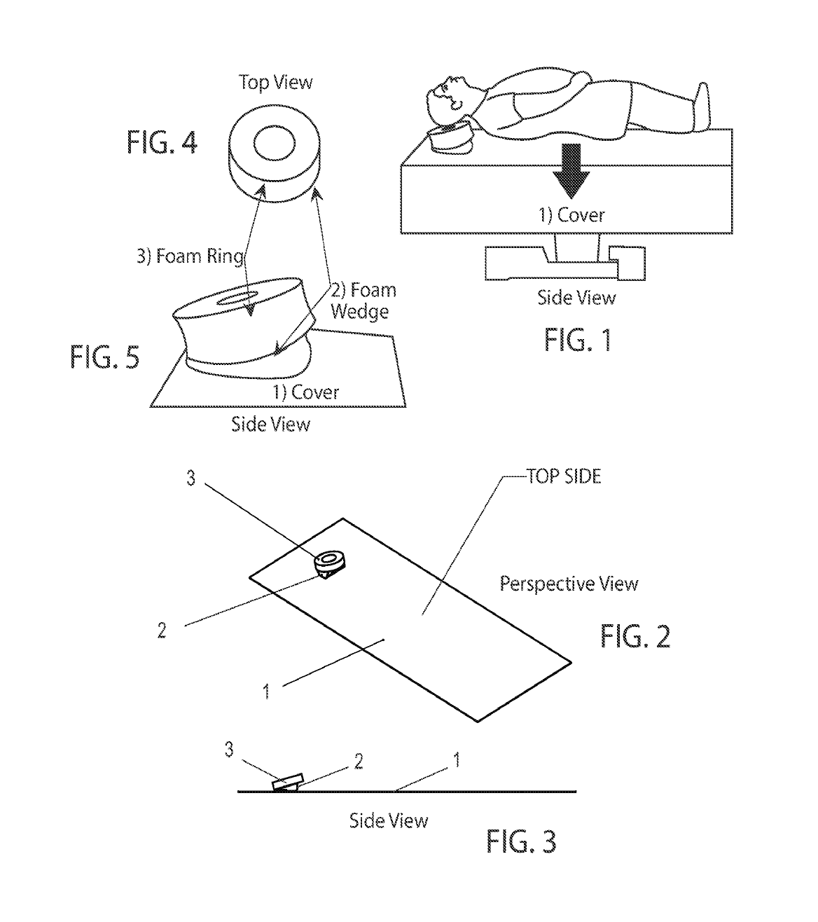

FIG. 1 is an exploded view, in perspective, of an OR table having a disposable barrier cover in accordance with one embodiment of the present invention;

FIG. 2 is a perspective view of a barrier cover of the present invention;

FIG. 3 is a side view of a barrier cover in accordance with the present invention;

FIG. 4 is a top view showing details of the head support portion of the OR table barrier cover in accordance with the present invention;

FIG. 5 is a side elevational view of the head support portion of the OR table disposable cover in accordance with the present invention;

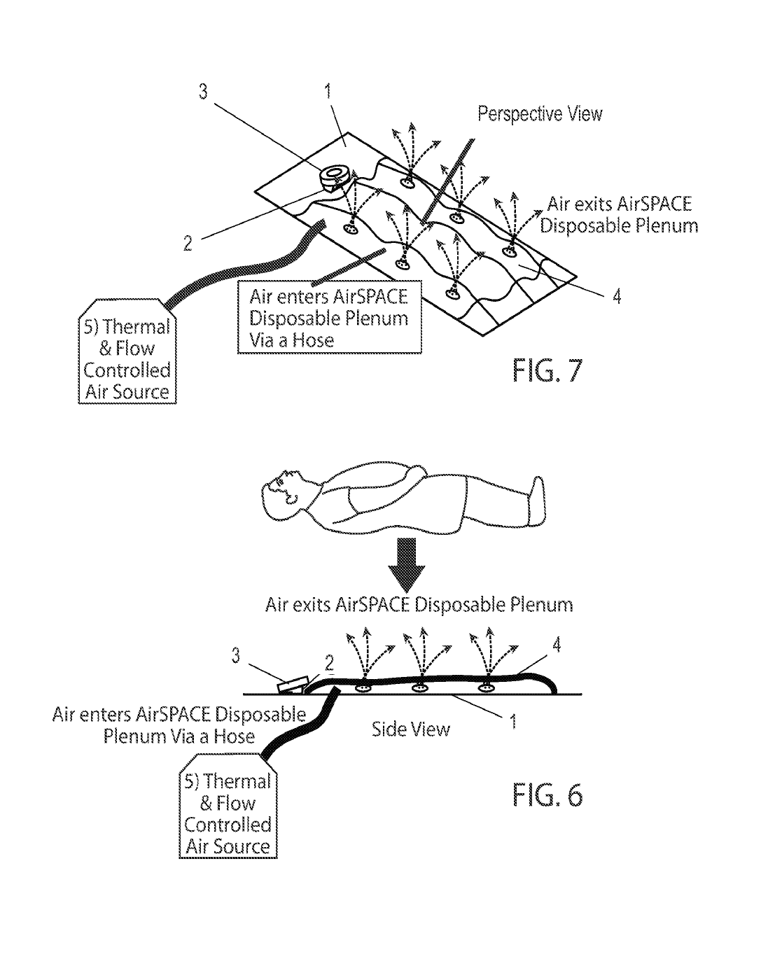

FIG. 6 is an exploded side elevational view of a second embodiment of OR table disposable cover in accordance with the present invention;

FIG. 7 is a perspective diagrammatic view of the FIG. 6 embodiment;

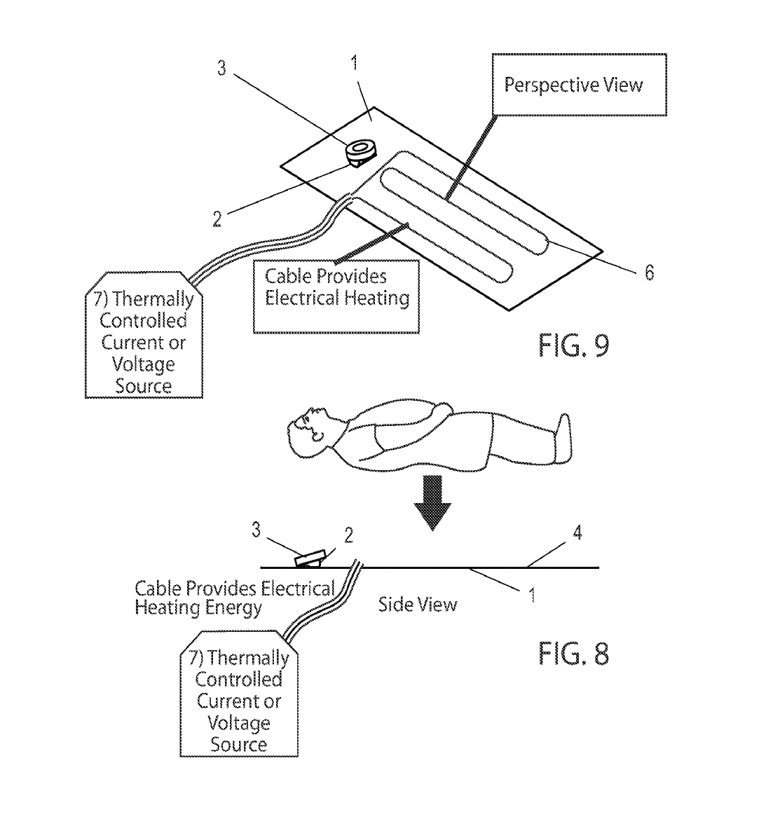

FIG. 8 is a view similar to FIG. 6 of a third embodiment of the OR table disposable cover in accordance with the present invention;

FIG. 9 is a view, similar to FIG. 7 of the third embodiment OR table disposable cover in accordance with the present invention;

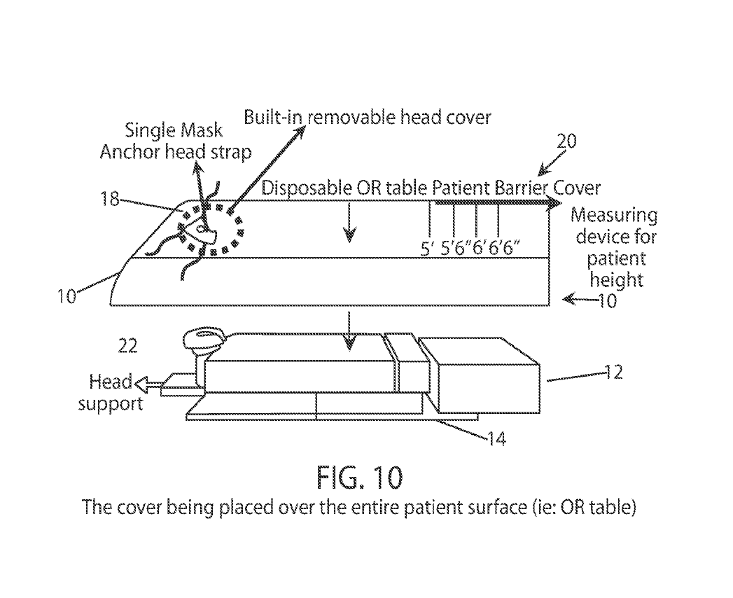

FIG. 10 is a perspective, partially exploded view of a OR table barrier cover being placed over an OR table in accordance with another embodiment of the present invention;

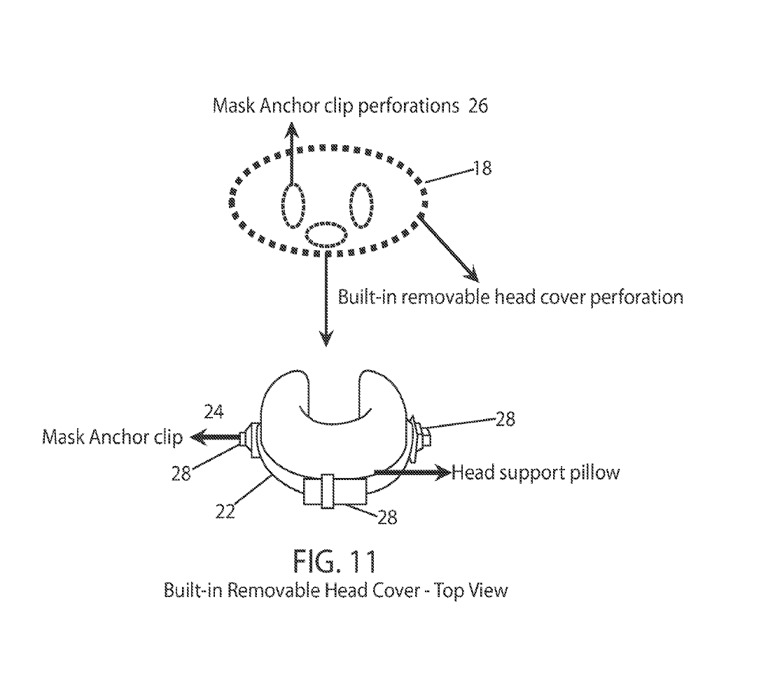

FIG. 11 is an exploded perspective view of a removable head cover portion of the barrier cover;

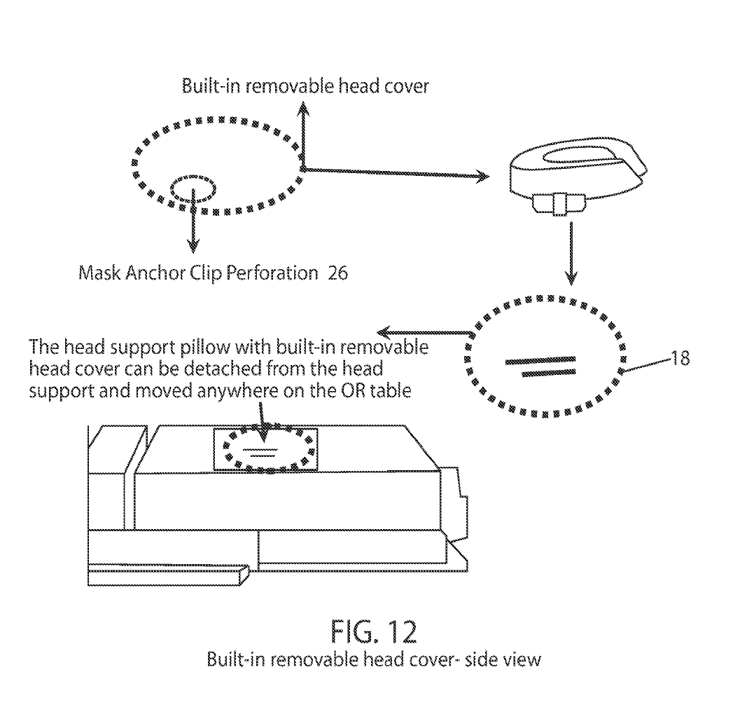

FIG. 12 is a schematic view illustrating how a head support pillow with a built-in removable head cover may be detached from the head support and moved elsewhere on an OR table;

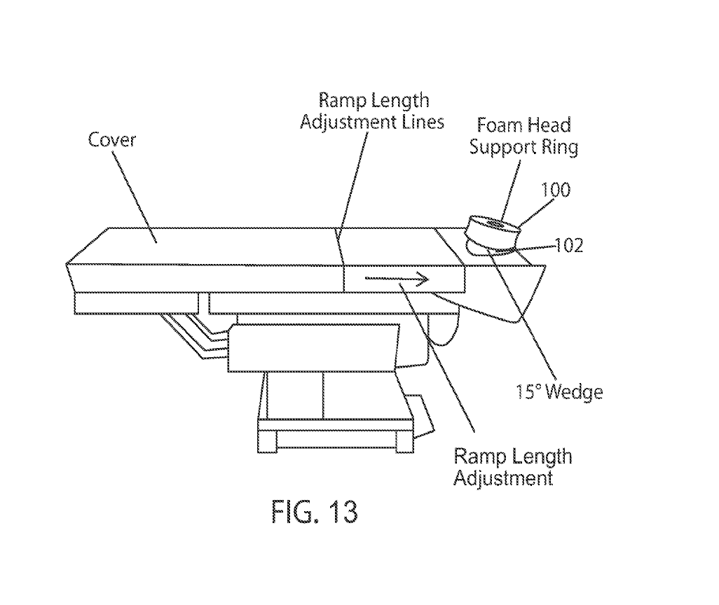

FIG. 13 is a view similar to FIG. 12 showing a OR table barrier cover in accordance with a second embodiment of the invention placed over the OR table;

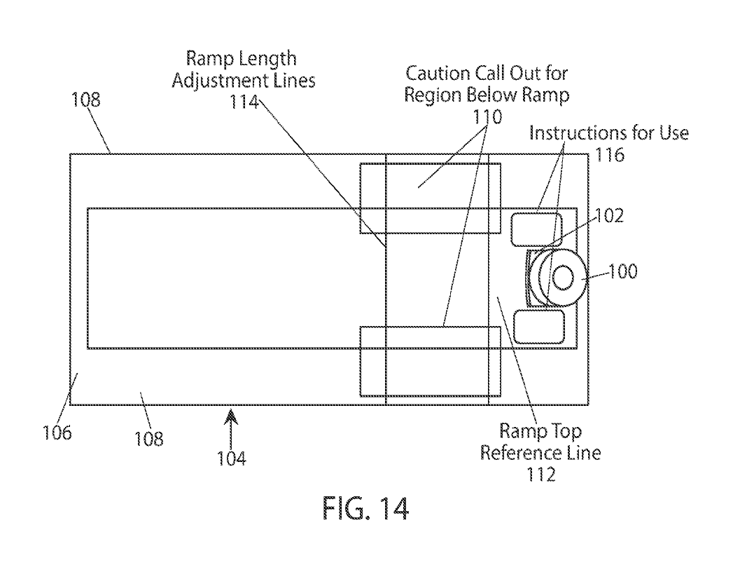

FIG. 14 is a top plan view of the OR table barrier cover in accordance with a second embodiment of the invention;

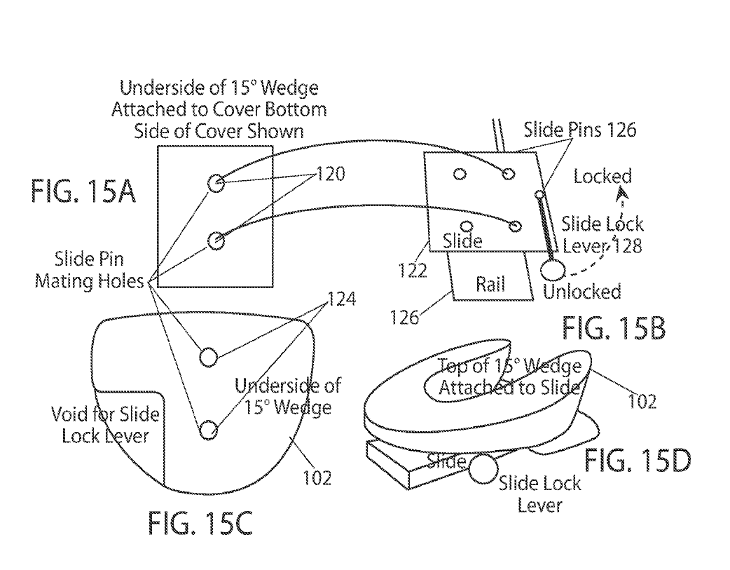

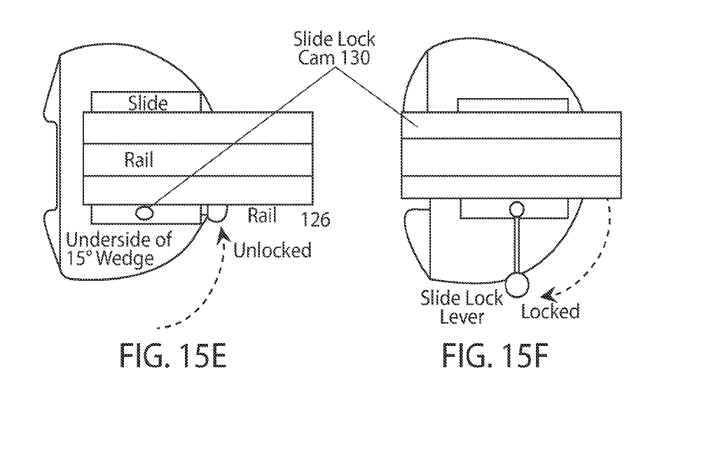

FIGS. 15A-15F show details of the wedge, wedge slide and slide lock mechanisms in accordance with a preferred embodiment of the present invention;

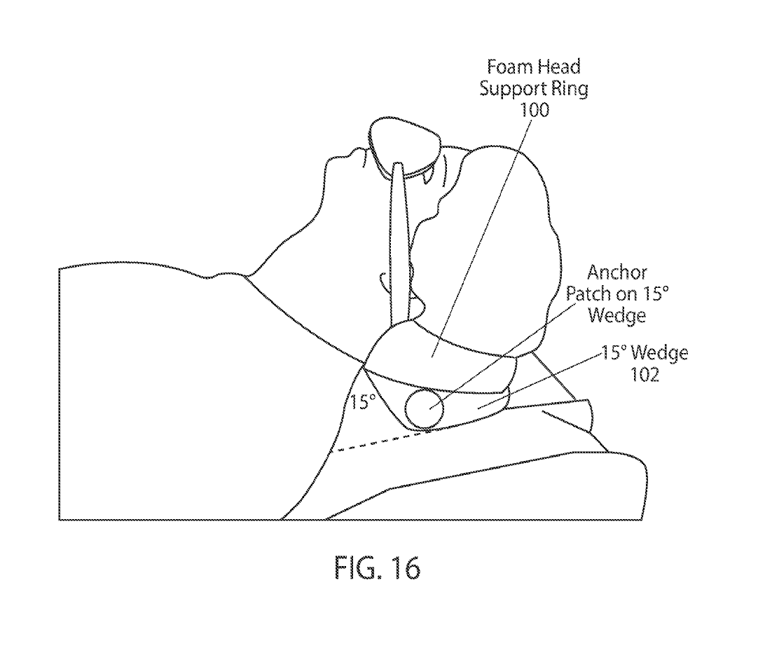

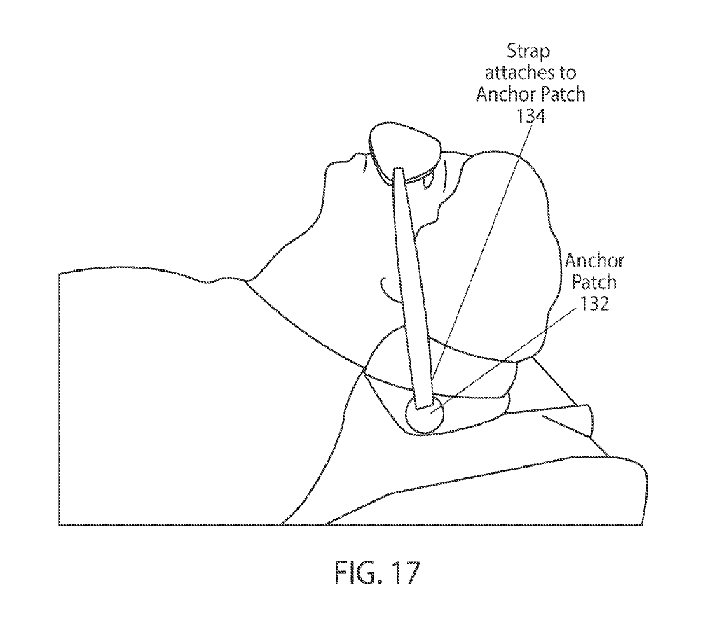

FIGS. 16 and 17 show a patient resting on a head supporting ring in accordance with the present invention;

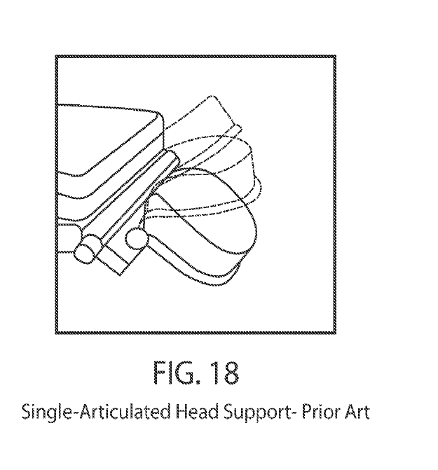

FIG. 18 shows a prior art single-articulated head support;

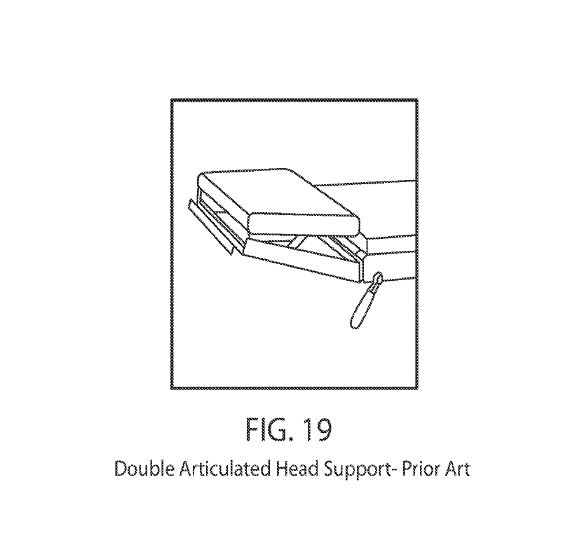

FIG. 19 shows a prior art double articulated head support;

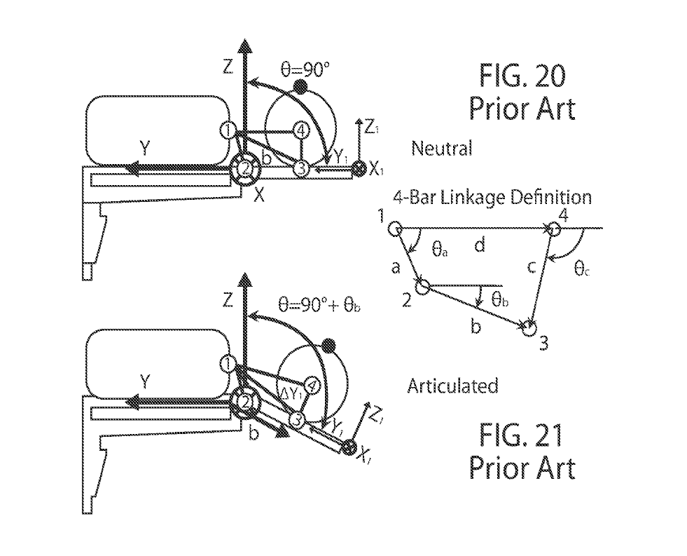

FIG. 20 illustrate a 4 bar linkage on a prior art single-articulated head support;

FIG. 21 illustrate a coordinate system for an OR table and a prior art single-articulated head support;

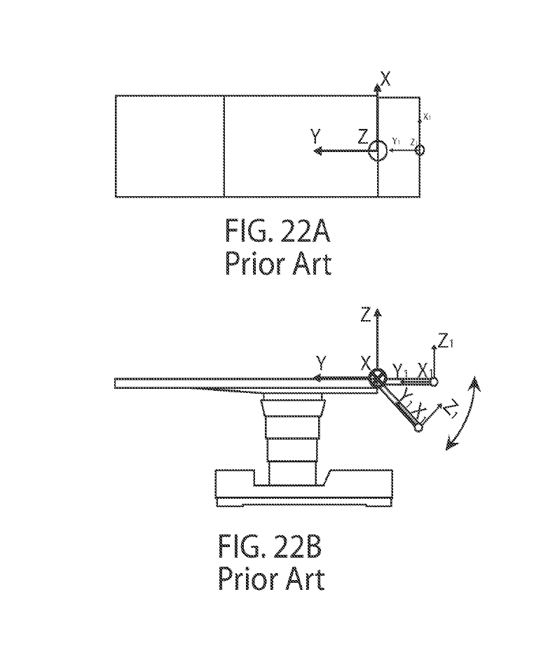

FIGS. 22A and 22B illustrate a coordinate system for an OR table with a prior art double articulated linkage geometry;

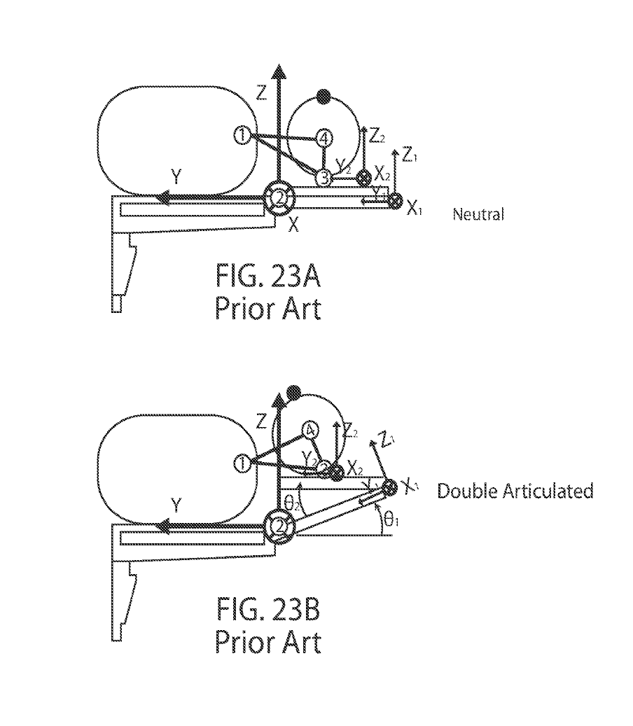

FIGS. 23A and 23B illustrate a rail and carriage head support system in accordance with embodiment of the present invention;

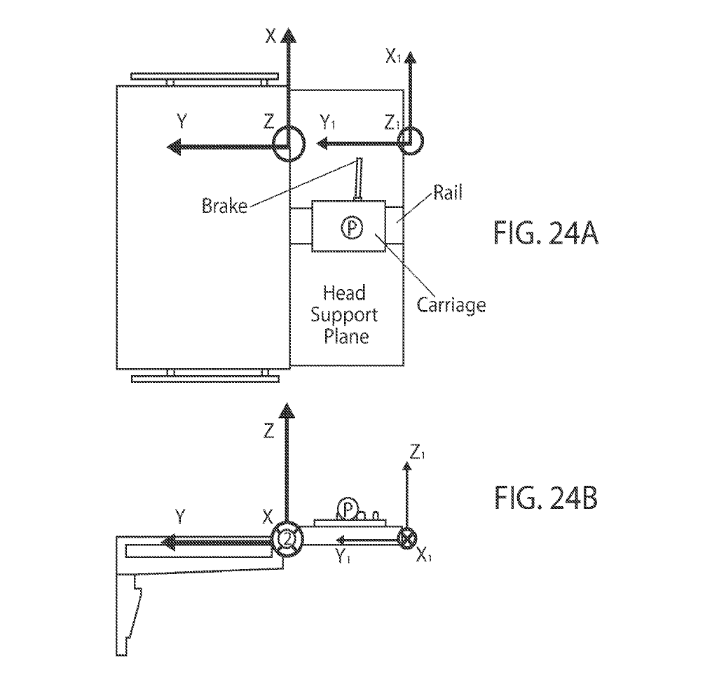

FIGS. 24A and 24B illustrate a head support cushion in accordance with the present invention;

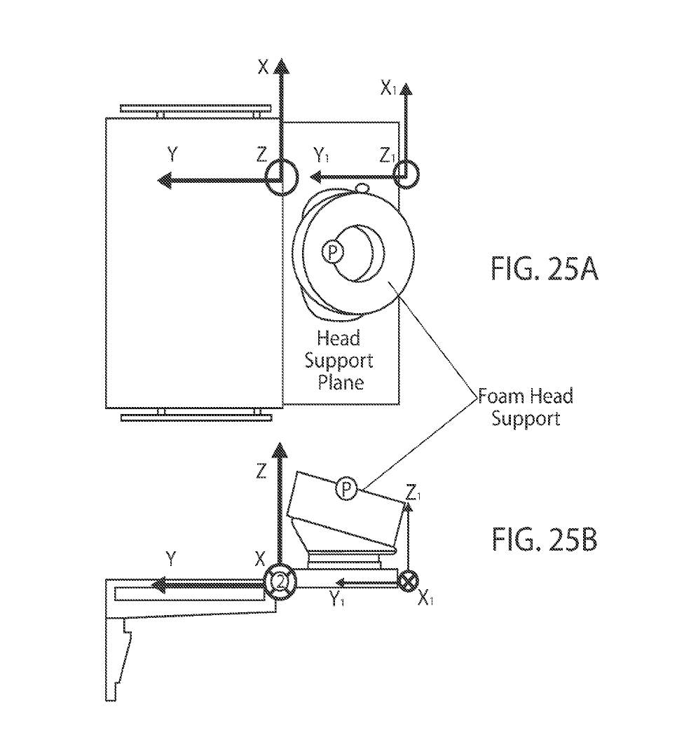

FIGS. 25A and 25B illustrate movement of a patient's head in accordance with the present invention; and

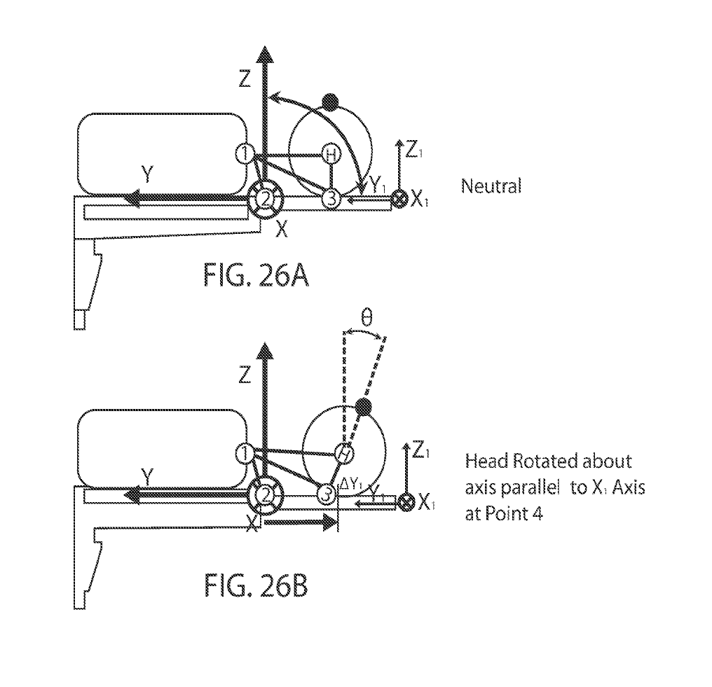

FIGS. 26A and 26B illustrate movement of a patient's head in accordance with the present invention.

As used herein, the term “OR table” is intended to include not only OR tables, but other patient supports including stretchers, gurneys and hospital beds, which are given as exemplary.

Referring to FIGS. 1-5, an OR table disposable cover is used to provide proper head support for a patient, absorb fluids from a procedure, and protect the operating table from those fluids. Major elements of the OR table disposable cover consist of the following elements:
Cover sheet 1, which is fluid absorbent on its top surface and fluid impermeable on its bottom surface;
An angled support wedge 2, preferably angled at approximately 15° to place the patient in a sniff position; and

A soft foam ring 3 interfering between the wedge 2 and the patient.

The OR table disposable barrier cover shown in FIGS. 1-5 may be used on a conventional OR table. However, to enjoy maximum advantages, the cover preferably is used in connection with an AIRSPACE™ patient positioning device available from Revolutionary Medical Devices, Inc. of Tucson, Ariz., as described in our aforesaid PCT/US14/44934 Application, or our PCT/US15/45357 Application.

Referring to FIGS. 6 and 7, in a second embodiment of the invention there is incorporated into an OR table disposable cover, as above described, a thermally controlled air flow source 5, supplying thermally controlled air flow into a plenum 4, which then exits the plenum at multiple locations between the plenum surface and patient. The temperature can be controlled either open loop, or closed loop with temperature sensors located at the air source or on the plenum at the patient interface, similar to commercially available patient warming products such as the 3M™ Bair Hugger™ Adult Underbody Blanket, Model 545

(http://solutions.3m.com/wps/portal/3M/en_US/IPD-NA/3M-Infection-Prevention/ products/catalog/˜/3M-Bair-Hugger-Adult-Underbody-Blanket-Model-545?N=7570550+3294264684&rt=d).

Alternatively, as shown in FIGS. 8-9, there is provided in accordance with a third embodiment of the invention an OR table disposable cover, an electrically resistive array 6, connected to a voltage and or current source 5. The temperature would be controlled either open loop, or closed loop with temperature sensors located at the air source or on the plenum at the patient interface, similar to commercially available patient warming products such as the Hot Dog (http://hotdog-usa.com/) produced by Augustine Temperature Management that utilizes electrical energy via a resistive array to provide patient heating.

Referring to FIGS. 10-12, a barrier cover 10 in accordance with another embodiment of the invention comprises a sheet material formed of a suitable material, typically impervious to fluid, and having a fluid absorbable top surface. The barrier cover includes a first (bottom) liquid imperious side that comes into contact with a patient support surface of a patient positioning device 12 such as described in our aforesaid PCT/US14/44934, supported on an OR table 14, stretcher, gurney or the like, or integrated as the patient supporting surface of an OR table, stretcher or gurney. The barrier cover 10 has a proximal end 16, with a built-in head cover 18, which is placed over and covers a head support surface for the patient. The head cover 18 is perforated so that it may be detached from the rest of the barrier cover 10. The second (second) side typically includes a liquid absorbing surface. Patient height measuring indicia 20 such as unit of length markings, e.g. Imperial, U.S. and/or metric markings are provided on the second side and are used to estimate the patient's height, and guide in patient placement in order to provide positioning on the patient positioning device 12. The cover 10 is placed over the entire patient positioning device 12 to drape over the OR table 14 and function to protect both the patient positioning device 12 and the OR table 14 from damage and protect the patient from cross contamination, etc.

The built-in removable head cover 18 is perforated so that it can be punched out from the barrier cover 10 and used to cover the head support 22 of the patient positioning device and protect it from blood, saline, etc. Referring to FIGS. 10 and 11, mask anchor head strap or straps may be attached to or detached from the head support 22 at anchors 24. The patient height-measuring indicia 20 are used to instruct the clinician on where to position a patient, and also to estimate how far the ramp as described in our aforesaid '934 PCT application should be extended for each patient. Indicia 20 also may include warnings or cautionary text or symbols to alert the clinician or other OR personnel of a potential pinching danger from the ramp mechanism.

Referring also to FIG. 12, one end of the barrier cover 10 contains a perforated built-in head cover 18, which may be removed, and protects and surrounds the head support 22. In a preferred embodiment the head cover 18 also has one or more pre-cut perforations or slots 26 for accommodating anesthesia head strap attachments, and, which allows the built-in removable head cover 18 to surround the anesthesia head strap attachments, such as but not limited to, spikes and cleats, and secures the built-in removable head support cover 18 to the head support 22. These pre-cut sections also allow anesthesia head strap attachment mechanisms 28 to be visible and used to attach all three anesthesia head strap arm extensions to the anesthesia head strap attachment mechanism. The built-in removable head cover 18 is pre-cut or perforated so it can be punched out of the patient barrier cover and placed over head support 22 should the head support 22 be moved elsewhere on the patient support surface, whereupon the head support 22 will still be covered.

Once the disposable assembly is placed on the device, the cover edges can be tucked under the cushions as shown in FIG. 13.

The disposable barrier assembly provides a protective barrier between the patient and the OR table and provides support to the patient's head, placing it in the sniff position. An alternative embodiment of disposable barrier assembly is shown in FIGS. 14-17. It consists of a soft foam head support ring 100 that the patient's head rests on, a 15° wedge 102 that places the patient in a sniff position and a main cover sheet 104. The top surface of the main cover sheet 104 is absorbent to fluids in. The bottom of the cover, not shown, is a liquid impermeable layer. The edge area 108 of the cover is either tucked under the OR table cushions or is draped over the side of the OR table. The foam head support ring 100 preferably is bonded to the 15° wedge 102 which in turn is bonded to the cover 104. Alternatively, wedge 102 may be eliminated, and the bottom of ring 100 cut at a 15° angle.

The top surface of cover 104 shown in particular in FIG. 14 has several items or indicia printed thereon. Caution pinch zone markings 110 on both sides of the cover provide notification that there is a hazard for pinching the operator or patient below the ramp. Indicia 112 provides a reference mark for the clinician to place the cover reference mark even with the top of the ramp. By placing the cover at this reference point, indicia 114 provides a reference line that the clinician should adjust the length of the ramp until the reference line that is close to the estimate height of the patient is aligned with the ramp pivot axis as illustrated in FIG. 13. Note that the approximate ratio of a patient's height to their hip joint to shoulder distance (where they are placed on the ramp) is 0.28-0.32 for men and women respectively. The distance from indicia 112, the top of ramp reference line and indicia 114, ramp length adjustment lines, preferably are equal to the patient's estimated height multiplied by 0.3. Indicia 116 provide instructions for use in order for a clinician to position the disposable assembly on top of the device and/or how to operate the device.

Referring to FIG. 15D, the bottom of the barrier cover 104 has two openings that allow for slide pin mating holes 120 to be exposed. The 15° wedge 102 is secured to a slide 122 on the head support of the device by forcing the mating holes 124 of the 15° wedge 102 onto the slide pins 126. An alternate or additional attachment configuration could have a hook & loop material interface on the top of the slide surface and bottom of the cover. The purpose of the slide is to allow the head position to translate along the long dimension on a rail 126 parallel to the length of the OR table as the OR table angles are adjusted. The clinician has the option of locking the slide to the rail by rotating a slide lock lever 128.

Details of the slide locking mechanism 128 are shown in 15E and 15F. When the lock lever 128 is parallel with the rail 126, the slide 122 is unlocked and can translate along the length of the rail 126. When the lock lever 128 is rotated 90°, the locking cam 130 engages the rail 126 through compressive force and friction and the slide is locked.

FIG. 16 shows a patient resting on the foam ring 100 and 15° wedge 102. The wedge 102 places the patient's neck in a 15° flexion angle in order to achieve the sniffing position. An anchor patch 132, shown in FIG. 16, is attached to the side of the 15° wedge 102. A second patch (not shown) is symmetrically located on the other side of the wedge. This patch can be a hook or loop material, where the mating strap 134 connected to each side of the mask secures the patient to the 15° wedge as shown in FIG. 17. The left strap is shown in FIG. 17 and the right strap, securing the right side is hidden.

Still another embodiment of the invention is discussed below.

Operating tables currently utilize single and double articulated head supports as shown in FIG. 18 and FIG. 19. See

(http://www.schaerermedical.ch/images/stories/zubehoerkataloge/accessories-e-s.pdf) and
(http://www.schaerermedical.ch/index.php?option=com_content&view=article&id=408&Itemid=116&lang=en). Problems with these arrant designs are twofold. First, the body, head & table interface is effectively a 4-bar linkage as illustrated in FIGS. 20 and 21 for a patient resting on a single-articulated head support. The 4-bar linkage consists of the patient's neck, vector d (1 to 4), the distance from the center of the head to where the head contacts the head support vector c (4 to 3), the distance from the base of the neck to the head support pivot point about the X axis vector a (1 to 2) and the distance along the head support vector b (2 to 4).

To first order, vectors a, d and c are known and constant, as is angle θ_{c}. As the head support is articulated about the X axis by angle θ_{b}, the b vector length and angle θₐ are reduced for positive θ_{b} and increased for negative θ_{b}. As a result, the head has to slide along the local Y₁ axis by an amount ΔY₁. The relationship between the vector length b and angle as a function θₐ of θ_{b} can be determined by solving for the following two equations simultaneously:

a cos(θₐ)+b cos(θ_{b})−c cos(θ_{c})−d=0 1.

a sin(θₐ)+b sin(θ_{b})−c sin(θ_{c})=0 2.

A problem with this design is that the patient's head is drug across the head support in the Y₁ direction when articulated, where the coordinate system for both the OR table, X, Y & Z and the single articulating head support X₁, Y₁ & Z₁ are shown in FIGS. 22A and 22B. The single articulating head support rotates about the X axis of the OR table. The double articulated head support shown in FIGS. 23A and 23B shares a similar problem when either of the support planes are rotated about the respective X or X₁ axis. (See FIGS. 22A and 22B).

Referring to FIGS. 24A and 24B, in order to overcome the aforesaid problem, a head support in accordance with the present invention includes a rail assembly that constrains a sliding head carriage in the X₁ and Z₁ direction, allowing it to slide along the Y₁ axis. The patients head would lay on the carriage corresponding to point P in the linkage diagrams. Additionally, there is a brake that allows the operator to fix the carriage along the Y₁ axis if needed.

It also is possible to have a carriage and rail configuration where a foam head support that cradles the patient's head is attached to the carriage, constrained in all degrees of freedom to the carriage as illustrated in FIGS. 25A and 25B.

Another substantial benefit of the rail and carriage with a brake is illustrated in FIGS. 26A and 26B. In order to intubate a patient, the anesthesiologist will rotate the patient's head about point H in order to place the patient in a sniff position. The rotation of an amount θ about point H parallel to the X₁ axis, will result in a translation along the Y₁ axis by ΔY₁ as shown. about point H parallel to the X₁ axis, will result in a translation along the Y₁ axis by ΔY₁ as shown. If the patient's head is placed on the carriage directly or on the foam support attached to the carriage, it can be rotated about point H parallel to the X₁ axis and when it is at the desired angel, the brake can be applied, setting the position of the carriage along the rail and holding the patient in the rotated position until the intubation is complete. The brake can then be released, allowing the head to relax in an original orientation about point H. While a single, articulated head support device is illustrated in FIGS. 24A-26B, a similar rail and sliding carriage device could be added to a double articulated device.

Various changes may be made in the above invention without departing from the spirit and scope thereof. For example, a similar rail and sliding carriage approach could be used in the support of other patient limbs, e.g. arms, legs, etc. where the equivalent of a 4-bar linkage is present.

1. An OR table disposable cover, comprising:
a sheet, fluid absorbent on a top side, and fluid impermeable on an opposite, bottom side;
a angled support wedge positioned in a head support region on the top side of the sheet; and
a soft foam member on a top of the angled support which in concert with its underlying support wedge maintains a patient's head in a sniff position.

2. The OR table disposable cover as claimed in claim 1, wherein the angle support wedge and foam member maintains a patient's head at approximately 15° angle relative to the OR table.

3. The OR table disposable cover as claimed in claim 1, wherein the angled support wedge is formed of a rigid foam.

4. The OR table disposable cover as claimed in claim 1, further including a patient warming device integrated with the OR table cover, said table cover including a plenum supplied with heated air from a thermally controlled air source and exiting a surface between the plenum and the patient, and further wherein the thermally-controlled heat source preferably operates in open loop, or closed loop with temperature feedback provided by temperature sensors located between the patient and plenum or at the thermally-controlled heat source.

5. The OR tale disposable cover as claimed in claim 1, further including a patient warming device integrated with the OR table disposable cover, said warming device including a resistive grid, and further wherein the resistive grid preferably operates in open loop, or closed loop with temperature feedback provided by temperature sensors located between the patient and electrical an current or voltage source.

6. A barrier cover having a removable head cover adjacent one end, and indicia for determining approximate patient height and for guiding location of a patient on a support surface.

7. The barrier cover of claim 6, wherein the removable head cover section has one or more pre-cut sections for permitting access to a patient head support for permitting attachment of mask anchor and/or head anchor straps.

8. The barrier cover of claim 6, characterized by one or more of the following features:
(a) wherein the removable head cover is perforated so that it may be punched out and moved elsewhere on the barrier cover;
(b) wherein the indicia are unit of length markings, e.g., Imperial, U.S. and/or metric markings; and
(c) wherein the removable head cover, has one or more anesthesia mask strap attachment mechanism, which allows the head cover to surround an anesthesia mask strap attachment mechanism, and further wherein the pre-cut sections preferably allow anesthesia head strap attachment mechanisms to be visible and used to attach to head straps.

9. A barrier cover having a patient head support member adjacent one end, wherein said head support member is canted at an about 15° angle to the cover.

10. The barrier cover of claim 9, characterized by one or more of the following features:
(a) wherein the head support member is formed of a resiliently deformable material, preferably a foam material;
(b) wherein the head support member is canted by a wedge support, and wherein the head support member preferably is bonded to the wedge support;
(c) wherein the head support member is fixed to the barrier cover, and wherein the head support member preferably is fixed to the barrier cover by hook and loop fasteners;
(d) wherein the head support member is adapted to be held on a table by posts; and
(e) further including a mask strap attachment mechanism for engaging with mask straps, wherein the mask strap attachment mechanism preferably comprises hook and loop.

11. A rail that is attached to an operating table head support, constraining a carriage in the X and Z axes, allowing a carriage to travel along the rail in a Y axis.

12. The rail as claimed in claim 11, further comprising, a brake that when engaged, holds the carriage in position on the rail.

13. An operating table carriage that interfaces with the patient either directly, or through a cushion that allows a patient's head to translate along an Y axis of the rail.

14. An operating table carriage as claimed in claim 13, further comprising, a brake that when engaged, holds the carriage in position on the rail.

15. The OR table disposable cover as claimed in claim 2, wherein the angled support wedge is formed of a rigid foam.

16. The OR table disposable cover as claimed in claim 2, further including a patient warming device integrated with the OR table cover, said table cover including a plenum supplied with heated air from a thermally controlled air source and exiting a surface between the plenum and the patient, and further wherein the thermally-controlled heat source preferably operates in open loop, or closed loop with temperature feedback provided by temperature sensors located between the patient and plenum or at the thermally-controlled heat source.

17. The OR tale disposable cover as claimed in claim 2, further including a patient warming device integrated with the OR table disposable cover, said warming device including a resistive grid, and further wherein the resistive grid preferably operates in open loop, or closed loop with temperature feedback provided by temperature sensors located between the patient and electrical an current or voltage source.

18. The barrier cover of claim 7, characterized by one or more of the following features:
(a) wherein the removable head cover is perforated so that it may be punched out and moved elsewhere on the barrier cover;
(b) wherein the indicia are unit of length markings, e.g., Imperial, U.S. and/or metric markings; and
(c) wherein the removable head cover, has one or more anesthesia mask strap attachment mechanism, which allows the head cover to surround an anesthesia mask strap attachment mechanism, and further wherein the pre-cut sections preferably allow anesthesia head strap attachment mechanisms to be visible and used to attach to head straps.
